(19) United States
(12) Patent Application Publication
(10) Pub. No.: US 2018/0098748 A1
(43) Pub. Date: Apr. 12, 2018

(54) LASER-INDUCED THERMAL STRAIN IMAGING SYSTEM AND METHOD USING IMPLANTABLE MEDICAL DEVICE, AND IMPLANTABLE MEDICAL DEVICE FOR LASER-INDUCED THERMAL STRAIN IMAGING

(71) Applicant: POSTECH ACADEMY-INDUSTRY FOUNDATION, Pohang-si (KR)

(72) Inventors: Chul-Hong KIM, Pohang-si (KR); Seong-Hee CHO, Busan (KR); Sung-Jo PARK, Gyeongsan-si (KR); Chang-Hoon CHOI, Pohang-si (KR)

(21) Appl. No.: 15/721,540

(22) Filed: Sep. 29, 2017

(30) Foreign Application Priority Data
Oct. 11, 2016 (KR) 10-2016-0131637

(51) Int. Cl. A61B 8/08; A61B 8/00

(52) U.S. Cl. CPC A61B 8/0858; A61B 8/587; A61B 8/5223; A61B 8/485

(57) ABSTRACT

Disclosed is a laser-induced thermal strain imaging system and method using an implantable medical device, and the implantable medical device for laser-induced thermal strain imaging, the system including: a light source module emitting a laser beam; a light concentrating module concentrating the laser beam emitted from the light source module through an objective lens so as to enable the laser beam to enter an optical fiber; an ultrasound signal obtaining module provided with a scanning stage and the device, the ultrasound signal obtaining module emitting the laser beam received through the optical fiber to an image area and obtaining an ultrasound signal generated from the area; a pulse generating and ultrasound signal receiving module generating an ultrasound pulse being transmitted to the ultrasound signal obtaining module, and receiving and amplifying the signal; and a data obtaining module obtaining a thermal strain image through an algorithm by using the signal.

### CROSS REFERENCE TO RELATED APPLICATION

The present application claims priority to Korean Patent Application No. 10-2016-0131637, filed Oct. 11, 2016, the entire contents of which is incorporated herein for all purposes by this reference.

### BACKGROUND OF THE INVENTION

### Field of the Invention

The present invention relates generally to a laser-induced thermal strain imaging system. More particularly, the present invention relates to a laser-induced thermal strain imaging system and method using an implantable medical device, and the implantable medical device for laser-induced thermal strain imaging.

### Description of the Related Art

Diagnosis using ultrasound and light can show a current diagnosis position and a cross-sectional image of tissue in real time, and by use thereof, the types, lengths, and states of the lesions can be quantitatively and qualitatively distinguished in three dimensions. However, these methods simply show the structure of tissue, and thus it is difficult to determine physiological information in the structure of the lesion. Typically, as an example, an ultrasound catheter is used in distinguishing intravascular atherosclerotic plaque. The ultrasound catheter shows the overall degree and structure of vascular stenosis, but is limited in distinguishing general atherosclerotic plaque from severe atherosclerotic plaque, which is a major cause of acute myocardial infarction and stroke.

In the meantime, a thermal strain imaging technique is a technique for imaging strain changes by measuring ultrasound in tissue right before and after applying heat to target biological tissue by using various heat sources. The technique is based on the fact that ultrasound velocity change as a consequence of temperature change differs depending on the type of biological tissue. The technique is typically used in noninvasively measuring the temperature change and in distinguishing tissue containing substantial lipids in the human body. Since biological tissue containing substantial water and biological tissue containing substantial lipids have opposite thermal strain changes, the strain changes in two tissues contrast when the temperature changes. Thus, the types of tissue may be distinguished by comparing the strain changes. However, low efficiency and sensitivity of the heat sources for changing the body temperature are major problems. An ultrasound system for measuring nuchal translucency using strain imaging modality is disclosed in Korean Patent No. 10-1194287, and medical ultrasound imaging is disclosed in Korean Patent Application Publication No. 10-2016-0056867.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose a laser-induced thermal strain imaging system and method using an implantable medical device, and the implantable medical device for laser-induced thermal strain imaging, the system, method, and device being capable of effectively transmitting a laser beam by transmitting the laser beam emitted from a light source module through an optical fiber and by directly emitting the laser beam to an image area, whereby it is possible to solve sensitivity and efficiency problems of a strain image, which occur due to difficulty in causing a temperature change in a body when using a conventional heat source.

Also, the present invention is intended to propose a laser-induced thermal strain imaging system and method using an implantable medical device, and the implantable medical device for laser-induced thermal strain imaging, the system, method, and device being capable of selectively rising the temperature of tissue in an image higher than another tissue by determining wavelength of a laser beam according to absorptance of an image area, namely, by selectively using the laser beam of a wavelength region where absorptance of target image tissue is high. Also, the system, method, and device are capable of effectively selecting particular tissue in the image, and of selectively obtaining an image according to component of tissue, whereby it is possible to obtain structural information as well as physiological information, which cannot be obtained from conventional ultrasound and optical images.

Also, the present invention is intended to propose a laser-induced thermal strain imaging system and method using an implantable medical device, and the implantable medical device for laser-induced thermal strain imaging, the system, method, and device enabling various sizes and applications by transmitting a laser beam through an optical fiber and by selectively using a laser beam according to absorptance of an image area. Also, the system, method, and device enable customized vascular catheter and endoscope to be manufactured such that medical devices can be actively adopted, and can effectively use a conventional method of obtaining a thermal strain image from outside of the body by using a laser beam.

In order to achieve the above object, according to one aspect of the present invention, there is provided a laser-induced thermal strain imaging system using an implantable medical device, the system including: a light source module emitting a laser beam; a light concentrating module concentrating the laser beam emitted from the light source module through an objective lens so as to enable the laser beam to enter an optical fiber; an ultrasound signal obtaining module provided with a scanning stage and the implantable medical device, the ultrasound signal obtaining module emitting the laser beam received through the optical fiber to an image area and obtaining an ultrasound signal generated from the image area; a pulse generating and ultrasound signal receiving module generating an ultrasound pulse being transmitted to the ultrasound signal obtaining module, and receiving the ultrasound signal obtained by the ultrasound signal obtaining module and amplifying the ultrasound signal; and a data obtaining module obtaining a thermal strain image through a predefined algorithm by using the ultrasound signal amplified by the pulse generating and ultrasound signal receiving module.

Preferably, the laser beam may have a wavelength determined according to absorptance of the image area.

Preferably, the light concentrating module may use a collimator to transmit the laser beam emitted from the light source module to the objective lens without being dispersed.

Preferably, the scanning stage may control rotation and pull-back of the implantable medical device.

Preferably, the implantable medical device may include: an ultrasound transducer obtaining the ultrasound signal generated from the image area; the optical fiber having an end provided at a side of the ultrasound transducer, the optical fiber emitting the laser beam emitted from the light source module to the image area; a pulse and ultrasound signal transmitting means coupled to the ultrasound transducer, the pulse and ultrasound signal transmitting means transmitting the ultrasound pulse generated by the pulse generating and ultrasound signal receiving module to the ultrasound transducer, and transmitting the ultrasound signal obtained by the ultrasound transducer to the pulse generating and ultrasound signal receiving module; and a housing composed of a corrosion-resistant material made of metal to protect the ultrasound transducer, the optical fiber, and the pulse and ultrasound signal transmitting means from outside.

Preferably, the implantable medical device may be covered with an ultra-thin chemical film to protect an inner structure thereof.

Preferably, the end of the optical fiber may be provided with a prism attached thereto or may be inclinedly cut so as to emit the laser beam to the image area.

Preferably, the ultrasound transducer may obtain a pre-emission ultrasound signal and a post-emission ultrasound signal, the pre-emission ultrasound signal being reflected from the image area by transmitting the ultrasound pulse to the image area before the laser beam is emitted, and the post-emission ultrasound signal being emitted from the image area by absorbing the laser beam after the laser beam is emitted.

Preferably, the data obtaining module may obtain the thermal strain image by color mapping thermal strain derived through the predefined algorithm by using the pre-emission ultrasound signal and the post-emission ultrasound signal, to an ultrasound image obtained by performing image processing on the post-emission ultrasound signal.

According to another aspect, there is provided a laser-induced thermal strain imaging method using an implantable medical device, the method including: (1) transmitting a laser beam emitted from a light source module through an optical fiber; (2) transmitting a ultrasound pulse generated by a pulse generating and ultrasound signal receiving module; (3) obtaining, by the implantable medical device, a pre-emission ultrasound signal reflected from an image area by transmitting the ultrasound pulse being transmitted at step (2), to the image area, and a post-emission ultrasound signal generated from the image area by emitting the laser beam being transmitted through the optical fiber at step (1), to the image area; and (4) obtaining, by a data obtaining module, a thermal strain image through image processing by using the pre-emission ultrasound signal and the post-emission ultrasound signal obtained at step (3).

Preferably, the laser beam at step (1) may have a wavelength determined according to absorptance of the image area.

Preferably, the implantable medical device may include: an ultrasound transducer obtaining an ultrasound signal generated from the image area; the optical fiber having an end provided at a side of the ultrasound transducer, the optical fiber emitting the laser beam emitted from the light source module to the image area; a pulse and ultrasound signal transmitting means coupled to the ultrasound transducer, the pulse and ultrasound signal transmitting means transmitting the ultrasound pulse generated by the pulse generating and ultrasound signal receiving module to the ultrasound transducer, and transmitting the ultrasound signal obtained by the ultrasound transducer to the pulse generating and ultrasound signal receiving module; and a housing composed of a corrosion-resistant material made of metal to protect the ultrasound transducer, the optical fiber, and the pulse and ultrasound signal transmitting means from outside.

Preferably, the implantable medical device may be covered with an ultra-thin chemical film to protect an inner structure thereof.

Preferably, the end of the optical fiber may be provided with a prism attached thereto or may be inclinedly cut so as to emit the laser beam to the image area.

Preferably, before step (4), the method may further include amplifying, by the pulse generating and ultrasound signal receiving module, the pre-emission ultrasound signal and the post-emission ultrasound signal obtained at step (3).

Preferably, step (4) may include: (4-1) obtaining an ultrasound image by using the post-emission ultrasound signal obtained at step (3); (4-2) deriving thermal strain through a predefined algorithm by using the pre-emission ultrasound signal and the post-emission ultrasound signal obtained at step (3); and (4-3) obtaining the thermal strain image by color mapping the thermal strain derived at step (4-2) to the ultrasound image obtained at step (4-1).

According to still another aspect, there is provided an implantable medical device for laser-induced thermal strain imaging, the device including: an ultrasound transducer obtaining an ultrasound signal generated from an image area; an optical fiber having an end provided at a side of the ultrasound transducer, the optical fiber emitting a laser beam emitted from an external light source to the image area; a pulse and ultrasound signal transmitting means coupled to the ultrasound transducer, the pulse and ultrasound signal transmitting means transmitting an ultrasound pulse generated by an external pulser-receiver to the ultrasound transducer, and transmitting the ultrasound signal obtained by the ultrasound transducer to the external pulser-receiver; and a housing composed of a corrosion-resistant material made of metal to protect the ultrasound transducer, the optical fiber, and the pulse and ultrasound signal transmitting means from outside.

Preferably, the implantable medical device may be covered with an ultra-thin chemical film to protect an inner structure thereof.

Preferably, the end of the optical fiber may be provided with a prism attached thereto or may be inclinedly cut so as to emit the laser beam to the image area.

Preferably, the ultrasound transducer may obtain a pre-emission ultrasound signal and a post-emission ultrasound signal, the pre-emission ultrasound signal being reflected from the image area by transmitting the ultrasound pulse to the image area before the laser beam is emitted, and the post-emission ultrasound signal being emitted from the image area by absorbing the laser beam after the laser beam is emitted.

According to the laser-induced thermal strain imaging system and method using the implantable medical device, and the implantable medical device for laser-induced thermal strain imaging, the laser beam emitted from the light source module can be transmitted by using the optical fiber and can be directly emitted to the image area, whereby the laser beam can be effectively transmitted. Thus, it is possible to solve sensitivity and efficiency problems of a strain image, which occur due to difficulty in causing a temperature change in the body when using a conventional heat source.

Also, according to the present invention, by determining the wavelength of the laser beam according to absorptance of the image area, namely, by selectively using the laser beam of a wavelength region where absorptance of target image tissue is high, the temperature of tissue in the image can be selectively raised higher than another tissue. Also, a particular tissue in the image can be effectively selected, and an image can be selectively obtained according to the component of tissue. Thus, it is possible to obtain structural information as well as physiological information, which cannot be obtained from conventional ultrasound and optical images.

Also, according to the present invention, by transmitting the laser beam through the optical fiber and by selectively using the laser beam according to absorptance of the image area, various sizes and applications are available, and a customized vascular catheter and endoscope can be manufactured such that medical devices can be actively adopted. Also, a conventional method of obtaining a thermal strain image from outside the body by using a laser beam can be effectively used.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a view schematically illustrating a laser-induced thermal strain imaging system using an implantable medical device according to an embodiment of the present invention;

FIG. 2 is a view illustrating a mechanical structure of a laser-induced thermal strain imaging system using an implantable medical device according to an embodiment of the present invention;

FIGS. 3A to 3D are views schematically illustrating configurations of an implantable medical device of a laser-induced thermal strain imaging system using the implantable medical device according to an embodiment of the present invention;

FIGS. 4A to 4D are views illustrating obtaining thermal strain of pig fat by a laser-induced thermal strain imaging system using an implantable medical device according to an embodiment of the present invention;

FIG. 5 is a view illustrating a process of obtaining a thermal strain image by a data obtaining module of a laser-induced thermal strain imaging system using an implantable medical device according to an embodiment of the present invention;

FIGS. 6A and 6B are views illustrating obtaining of an ultrasound image by a laser-induced thermal strain imaging system using an implantable medical device according to an embodiment of the present invention;

FIGS. 7A to 7C are views illustrating obtaining of a 2D thermal strain image by a laser-induced thermal strain imaging system using an implantable medical device according to an embodiment of the present invention;

FIG. 8 is a flowchart illustrating a laser-induced thermal strain imaging method using an implantable medical device according to an embodiment of the present invention;

FIG. 9 is a flowchart illustrating a laser-induced thermal strain imaging method using an implantable medical device according to another embodiment of the present invention; and

FIG. 10 is a flowchart illustrating a laser-induced thermal strain imaging method using an implantable medical device according to still another embodiment of the present invention.

### DETAILED DESCRIPTION OF THE INVENTION

Hereinbelow, exemplary embodiments of the present invention will be described in detail with reference to the accompanying drawings such that the present invention can be easily embodied by one of ordinary skill in the art to which this invention belongs. In the following description of the present invention, detailed descriptions of known functions or components incorporated herein will be omitted when it may make the subject matter of the present invention unclear. Also, the elements having the similar functions and operations of the drawings are given the same reference numerals.

It will be understood that when a portion is referred to as being “connected” or “coupled” to another portion, it can be directly connected or coupled to the other portion or intervening portions may be present. Also, it should also be understood that when a component “includes” an element, unless there is another opposite description thereto, the component does not exclude another element but may further include the other element.

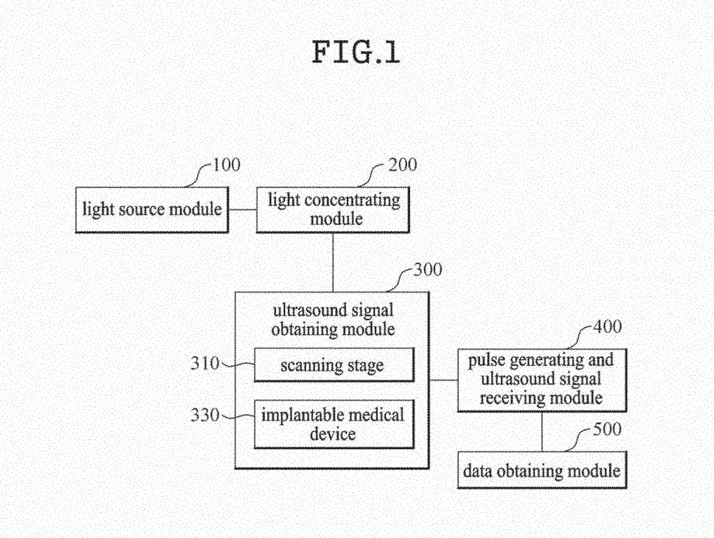

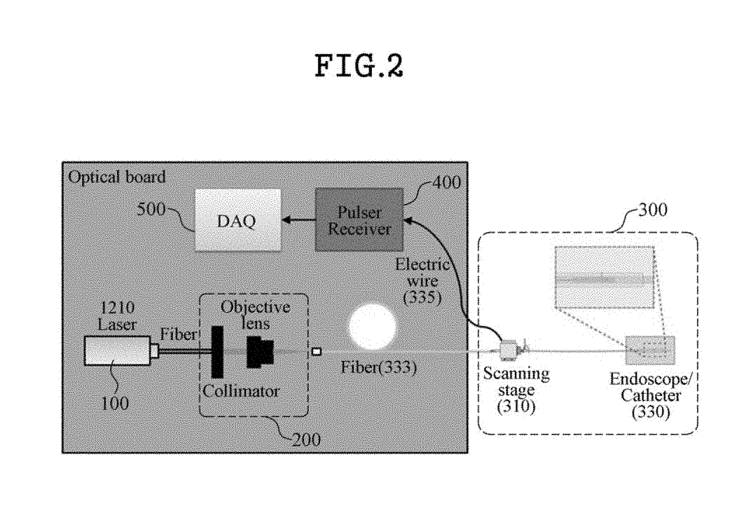

FIG. 1 is a view schematically illustrating a laser-induced thermal strain imaging system using an implantable medical device 330 according to an embodiment of the present invention. FIG. 2 is a view illustrating a mechanical structure of a laser-induced thermal strain imaging system using an implantable medical device 330 according to an embodiment of the present invention. As shown in FIGS. 1 and 2, according to the embodiment of the present invention, the laser-induced thermal strain imaging system using the implantable medical device 330 may include a light source module 100, a light concentrating module 200, an ultrasound signal obtaining module 300, a pulse generating and ultrasound signal receiving module 400, and a data obtaining module 500.

The light source module 100 may emit a laser beam. That is, the laser beam may be emitted to obtain an ultrasound signal of an image area irradiated by the laser beam. Here, the laser beam emitted from the light source module 100 has a wavelength that may be determined according to absorptance of the image area. Specifically, the laser beam of wavelength region where absorptance of an image area is high may be used.

As described above, by selectively using the laser beam of a wavelength region where absorptance of an image area is high, the temperature of the tissue in the image can be selectively risen higher than another tissue, particular tissue in the image can be effectively selected, and an image can be selectively obtained according to the component of tissue. Thus, it is possible to obtain structural information as well as physiological information that cannot be obtained from conventional ultrasound and optical images.

The light concentrating module 200 may concentrate the laser beams emitted from the light source module 100 through an objective lens such that the laser beams enter an optical fiber 333. Specifically, a collimator is used to transmit the laser beam emitted from the light source module 100 to the objective lens without being dispersed. The objective lens may concentrate the laser beam to the optical fiber 333. That is, the light concentrating module 200 may include a collimator and an objective lens.

In the meantime, the laser beam may be transmitted from the light source module 100 to the light concentrating module 200 through the optical fiber or a free space according to the embodiment.

The ultrasound signal obtaining module 300 may include a scanning stage 310 and an implantable medical device 330, and may emit the laser beam received through the optical fiber 333 to the image area, and may obtain an ultrasound signal generated from the image area.

Here, the scanning stage 310 may control rotation and pull-back of the implantable medical device 330, whereby 2D and 3D ultrasound images can be obtained. Here, the optical fiber 333 is coupled to the end portion of the implantable medical device 330 by passing through the scanning stage 310, and may emit the laser beam to the image area.

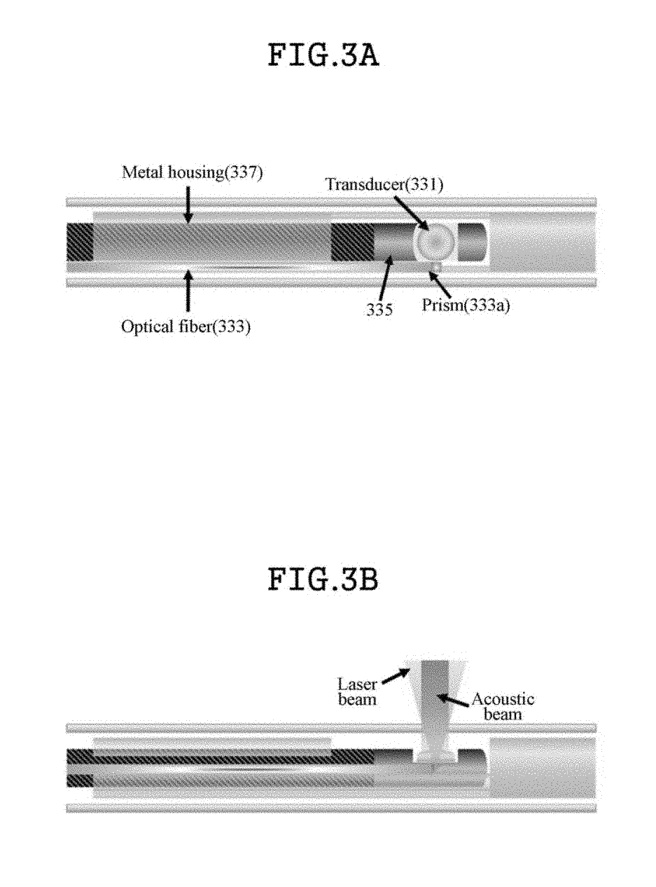

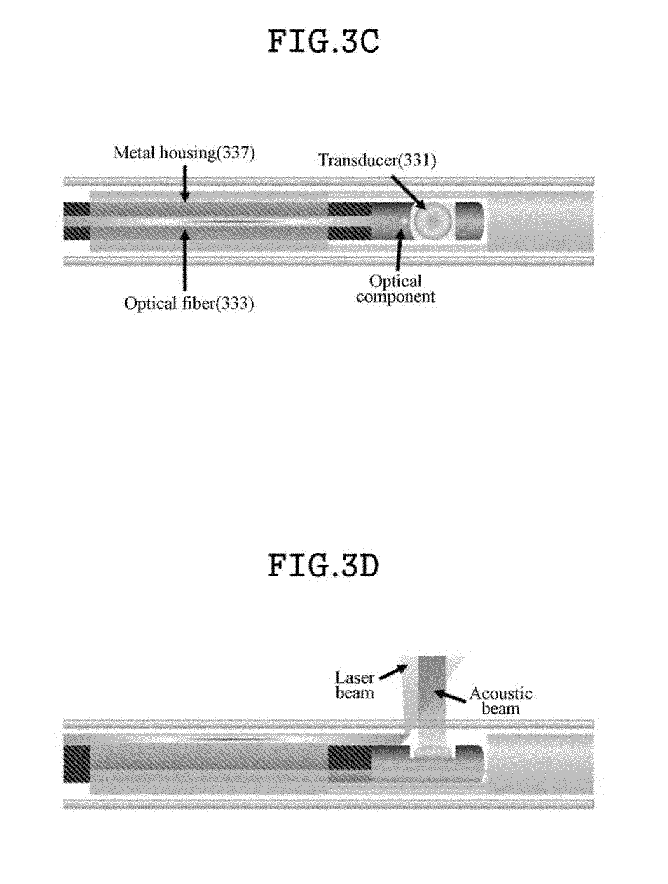

In the meantime, configuration of the implantable medical device 330 will be described in detail with reference to FIGS. 3A to 3D.

The pulse generating and ultrasound signal receiving module 400 may generate an ultrasound pulse being transmitted to the ultrasound signal obtaining module 300, and may receive the ultrasound signal obtained by the ultrasound signal obtaining module 300 and may amplify the ultrasound signal.

According to the embodiment, the pulse generating and ultrasound signal receiving module 400 may be realized by a general pulser-receiver.

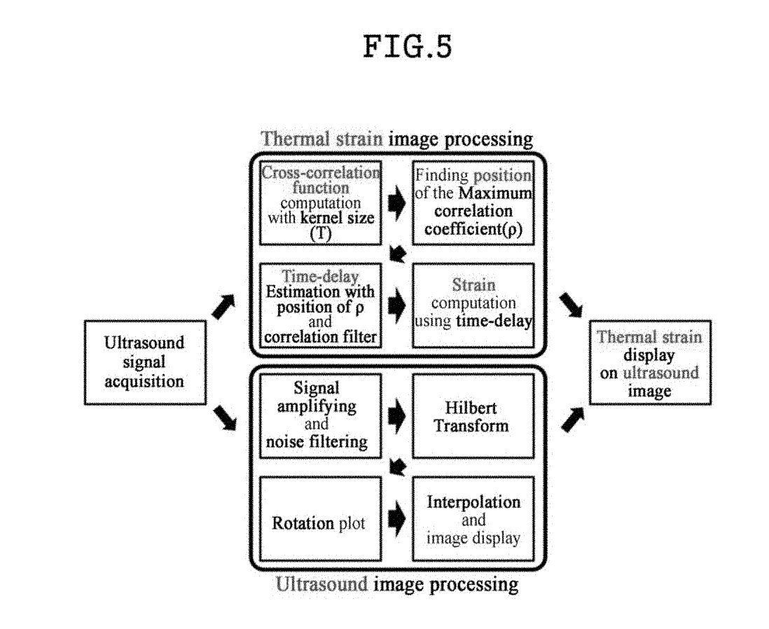

The data obtaining module 500 may obtain a thermal strain image through a predefined algorithm by using the ultrasound signal amplified by the pulse generating and ultrasound signal receiving module 400. Detailed configurations of the data obtaining module 500 will be described later with reference to FIGS. 3A to 3D, and 5.

In the meantime, using the laser beam of 1210 nm is illustrated in FIG. 2 for convenience of explanation, but the wavelength of the laser beam is not limited thereto, and a customized laser beam of various wavelengths may be used depending on target tissue in obtaining an image.

FIGS. 3A to 3D are views schematically illustrating configurations of an implantable medical device 330 of a laser-induced thermal strain imaging system using the implantable medical device 330 according to an embodiment of the present invention. Specifically, FIG. 3A is a plan view illustrating the implantable medical device 330, and FIG. 3B is a side view illustrating the implantable medical device 330. As shown in FIGS. 3A to 3D, the implantable medical device 330 of the laser-induced thermal strain imaging system using the implantable medical device 330 according to the embodiment of the present invention may include an ultrasound transducer 331, an optical fiber 333, a pulse and ultrasound signal transmitting means 335, and a housing 337.

Also, the implantable medical device may be covered with an ultra-thin chemical film to protect the inner structure thereof. Specifically, the ultra-thin chemical film is a material where attenuation of light and sound wave are small, and may be Pebax material according to the embodiment.

The ultrasound transducer 331 may obtain the ultrasound signal generated from the image area. Also, the ultrasound transducer 331 may obtain the ultrasound signal reflected from the image area.

Specifically, the ultrasound transducer 331 may obtain a pre-emission ultrasound signal reflected from the image area by transmitting the ultrasound pulse to the image area before the laser beam is emitted, and may obtain a post-emission ultrasound signal emitted from the image area by absorbing the laser beam after the laser beam is emitted. That is, the ultrasound transducer 331 may obtain both ultrasound signals before and after emitting the laser beam.

Here, the ultrasound pulse transmitted to the image area in order to obtain the pre-emission ultrasound signal may be generated by and received from the pulse generating and ultrasound signal receiving module 400.

Also, the data obtaining module 500 may obtain the thermal strain image by color mapping thermal strain derived through a predefined algorithm by using the pre-emission ultrasound signal and the post-emission ultrasound signal, to the ultrasound image obtained by performing image processing on the post-emission ultrasound signal obtained by the ultrasound transducer 331.

Here, the data obtaining module 500 may receive the pre-emission ultrasound signal and the post-emission ultrasound signal from the pulse generating and ultrasound signal receiving module 400, and may use the pre-emission ultrasound signal and the post-emission ultrasound signal for image processing.

In the meantime, the algorithm used for obtaining the thermal strain image by the data obtaining module 500 will be described in detail later with reference to FIG. 5.

The optical fiber 333 has an end provided at a side of the ultrasound transducer 331 such that the laser beam emitted from the light source module 100 may be emitted to the image area. Specifically, the optical fiber 333 may be fixed at a position that does not disturb the sound wave path of the ultrasound transducer 331.

Also, the optical fiber 333 may be provided to be collinear with the ultrasound transducer 331 as shown in FIGS. 3C (plan view) and 3D (side view).

That is, the optical fiber 333 may be coupled to the end portion of the implantable medical device 330 where the ultrasound transducer 331 is provided by passing through the scanning stage 310, and may emit the laser beam concentrated by the light concentrating module 200 after being emitted from the light source module 100, to the image area.

Here, the optical fiber 333 may have an end to which a prism 333a is attached or on which inclined cutting is performed so as to emit the laser beam to the image area. As described above, the end is attached with the prism 333a or inclined cutting is performed on the end, whereby the laser beam can be emitted at a consistent angle.

Also, without being limited thereto, according to the embodiment, the end of the optical fiber 333 may be provided with an optical component attached thereto, such as a GRIN lens and a ball lens, or may be realized as a lens optical fiber, a special optical fiber, etc. That is, any optical fiber may be used as long as it can modify the shape and the path of the laser beam.

As described above, the laser beam emitted from the light source module 100 can be transmitted by using the optical fiber 333 and can be directly emitted to the image area, whereby the laser beam can be effectively transmitted. Thus, it is possible to solve sensitivity and efficiency problems of a strain image, which occur due to difficulty in causing temperature change in the body when using a conventional heat source.

The pulse and ultrasound signal transmitting means 335 is coupled to the ultrasound transducer 331 such that the ultrasound pulse generated by the pulse generating and ultrasound signal receiving module 400 may be transmitted to the ultrasound transducer 331 and the ultrasound signal obtained by the ultrasound transducer 331 may be transmitted to the pulse generating and ultrasound signal receiving module 400.

The pulse and ultrasound signal transmitting means 335 may be realized by a general electric wire, and may be wrapped with a protective coil to protect the ultrasound signal being transmitted.

In the meantime, the protective coil wrapping the pulse and ultrasound signal transmitting means 335 may be coupled to the scanning stage 310. Here, the optical fiber 333 may be coupled to the scanning stage 310 with the pulse and ultrasound signal transmitting means 335 via the protective coil, or may be coupled to outside of the protective coil.

That is, the optical fiber 333 and the pulse and ultrasound signal transmitting means 335 may be respectively coupled to the implantable medical device 330 from the light concentrating module 200 and the pulse generating and ultrasound signal receiving module 400 via the scanning stage 310.

The housing 337 may be composed of a corrosion-resistant material made of metal to protect the ultrasound transducer 331, the optical fiber 333, and the pulse and ultrasound signal transmitted means 335 from outside.

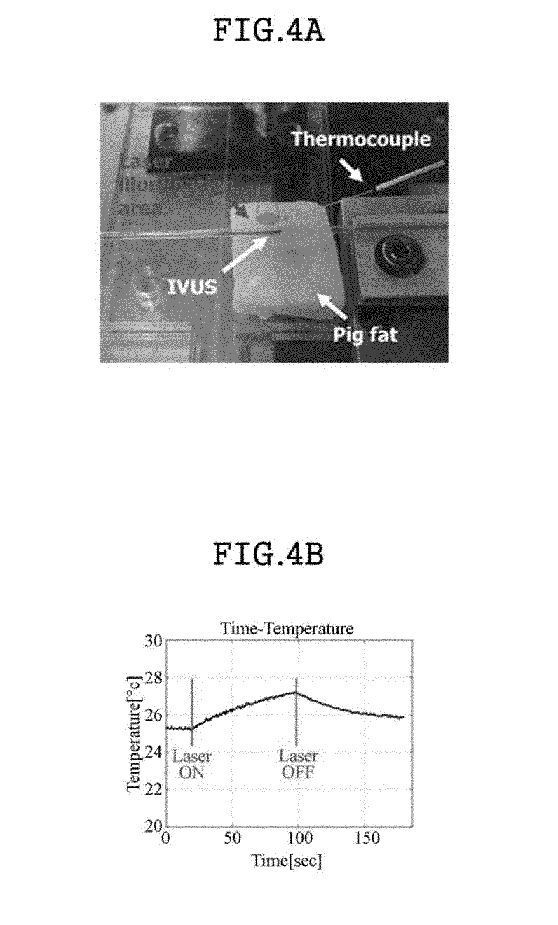

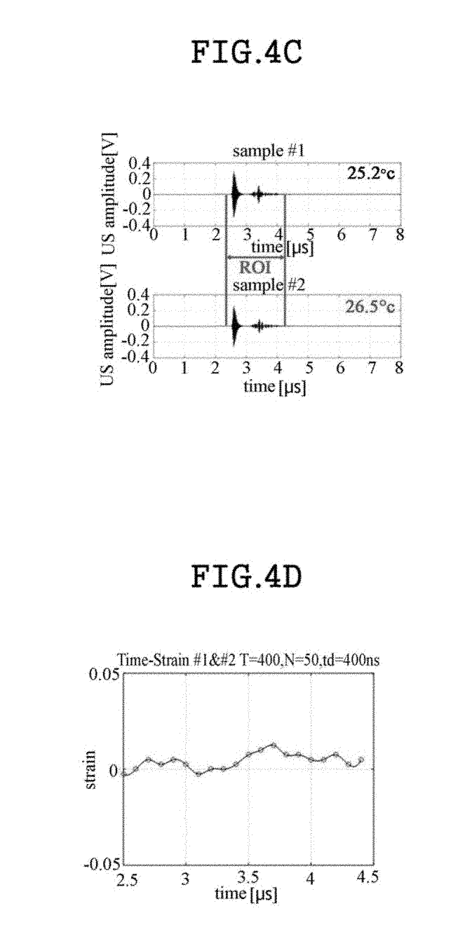

FIGS. 4A to 4D are views illustrating obtaining thermal strain of pig fat by a laser-induced thermal strain imaging system using an implantable medical device 330 according to an embodiment of the present invention. As shown in FIGS. 4A to 4D, by the laser-induced thermal strain imaging system using the implantable medical device 330 according to the embodiment of the present invention, thermal strain of pig fat can be effectively obtained.

Specifically, FIG. 4A is a view illustrating setting of pig fat temperature rise experiment. FIG. 4A shows setting of preliminary experiment where temperature change measurement and thermal strain signal measurement are performed by emitting the laser beam to pig fat, the laser beam having the spectral region of 1210 nm that is strongly absorbed into pig fat. As shown in FIG. 4A, in order to measure the temperature change of pig fat, a probe-type thermocouple is inserted into the pig fat, and the laser beam is emitted to the upper portion of the pig fat through the optical fiber 333. Also, the intravascular ultrasound (IVUS) in a transparent tube is fixed at the upper portion of the portion irradiated with the laser beam so as to consistently obtain the signal, and the experiment is performed in a water-filled tank.

FIG. 4B is a view illustrating the temperature change measurement of pig fat in consequence of emitting the laser beam. The power of the laser beam has intensity (1 W/cm²) similar to limited intensity of ANSI Laser Safety Standard NIR region. As shown in FIG. 4B, after waiting time about 20 seconds, the temperature of the pig fat rises when the laser beam starts to be emitted. When emission of the laser beam is stopped at 100 seconds, the temperature rise is stopped and the temperature is gradually lowered.

FIG. 4C is a view illustrating the ultrasound signal measured in consequence of the temperature change of the pig fat. A region near the start and end points of the ultrasound signal reflected from the pig fat is designated as a region of interest (ROI), and strain is measured by using time-delay change rate through a thermal strain algorithm.

FIG. 4D is a graph illustrating measured strain in ROI of the ultrasound signal obtained by using the pig fat. Here, the kernel size is designated as T, the filter coefficient is designated as N, and the time difference during strain measurement is designated as td. Theoretically, in a case of fat, when the temperature rises, the velocity of the ultrasound tends to decrease in proportion to the temperature rise, and strain tends to increase. Specifically, when the temperature rises by one degree, strain of about 0.0013 to 0.002 (0.13% to 0.2%) is measured at the fat. As shown in FIGS. 4B and 4C, the temperature rises about one to three degrees, and strain is measured as being between about 0.005 and 0.01, which is close to a theoretical value.

That is, according to the laser-induced thermal strain imaging system of the present invention, thermal strain corresponding to the theoretical value can be effectively derived, and a thermal strain image can be obtained through image processing.

FIG. 5 is a view illustrating a process of obtaining a thermal strain image by a data obtaining module 500 of a laser-induced thermal strain imaging system using an implantable medical device 330 according to an embodiment of the present invention. As shown in FIG. 5, the data obtaining module 500 of the laser-induced thermal strain imaging system using the implantable medical device 330 according to the embodiment of the present invention may derive thermal strain through the predefined algorithm, and may obtain the thermal strain image by color mapping the thermal strain to the ultrasound image.

Specifically, the ultrasound image may be obtained by performing image processing on the obtained post-emission ultrasound signal through Hilbert transform, rotation plot, and interpolation processes after signal amplification and noise reduction.

Also, a cross-correlation function may be applied by using the predetermined kernel size (T), the pre-emission ultrasound signal and the post-emission ultrasound signal. The time-delay of each point of two signals may be obtained by calculating the lag position of the maximum correlation coefficient. The thermal strain may be derived by using the time-delay. The derived thermal strain may be color mapped to the obtained ultrasound image. Consequently, the thermal strain image can be obtained.

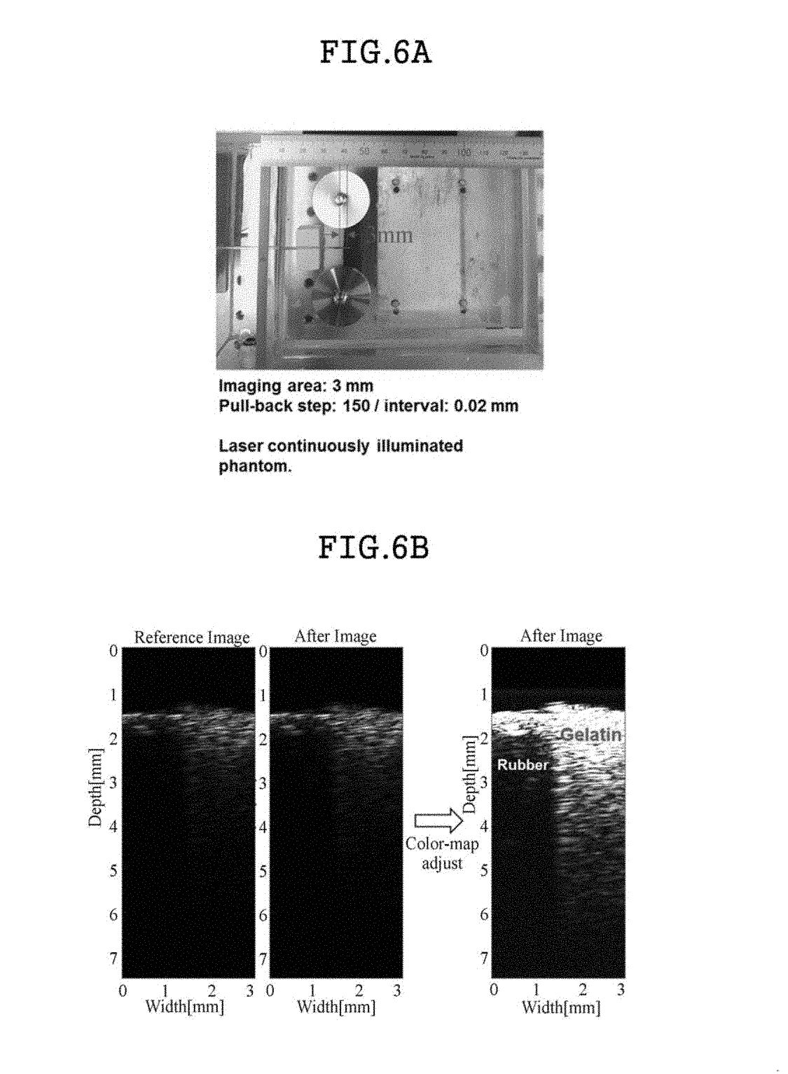

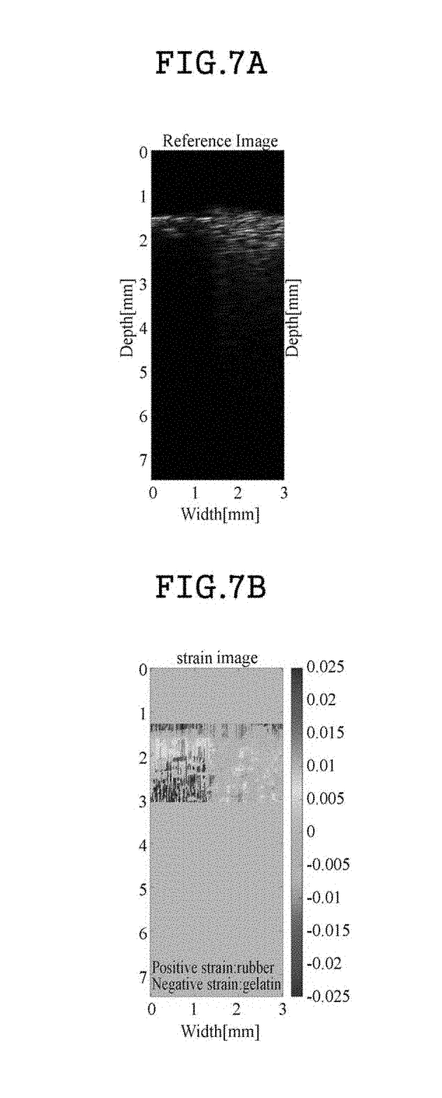

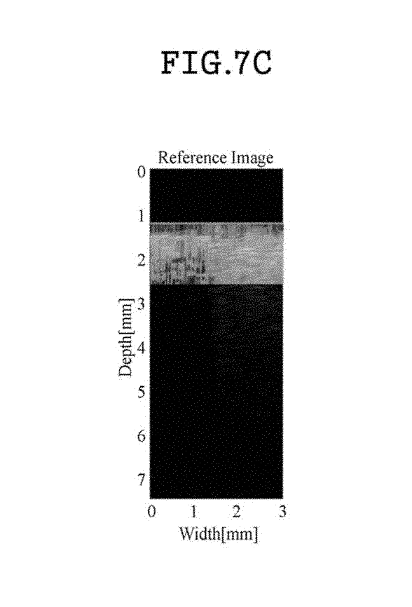

FIGS. 6A and 6B are views illustrating obtaining of an ultrasound image by a laser-induced thermal strain imaging system using an implantable medical device according to an embodiment of the present invention. FIGS. 7A to 7C is a view illustrating obtaining of a 2D thermal strain image by a laser-induced thermal strain imaging system using an implantable medical device according to an embodiment of the present invention. As shown in FIGS. 6A to 7C, according to the laser-induced thermal strain imaging system using the implantable medical device according to the embodiment of the present invention, the thermal strain image may be easily obtained by using the ultrasound image.

Specifically, the left and the middle images of FIG. 6B respectively show ultrasound images before and after emitting the laser beam to the experimental tissue. The experimental tissue is formed by attaching gelatin and rubber, and the laser beam has the wavelength region where the rubber absorbs relatively more. The right image of FIG. 6B may be obtained by applying color mapping to the ultrasound image of the middle image of FIG. 6B obtained after emitting the laser beam. As shown in the right image of FIG. 6B, the boundary between the rubber and the gelatin is clearly distinguished in the ultrasound image. Here, the right image of FIG. 6B may be used in obtaining the thermal strain image.

FIG. 7A shows an image before emitting the laser beam, FIG. 7B shows an thermal strain image obtained by using the predefined algorithm, and FIG. 7C shows the ultrasound image after emitting the laser beam overlaid with the thermal strain image of FIG. 7B, namely, an image obtained by color mapping thermal strain on the ultrasound image. As shown in FIGS. 7A to 7C, strain significantly changes at the rubber that significantly absorbs the laser beam.

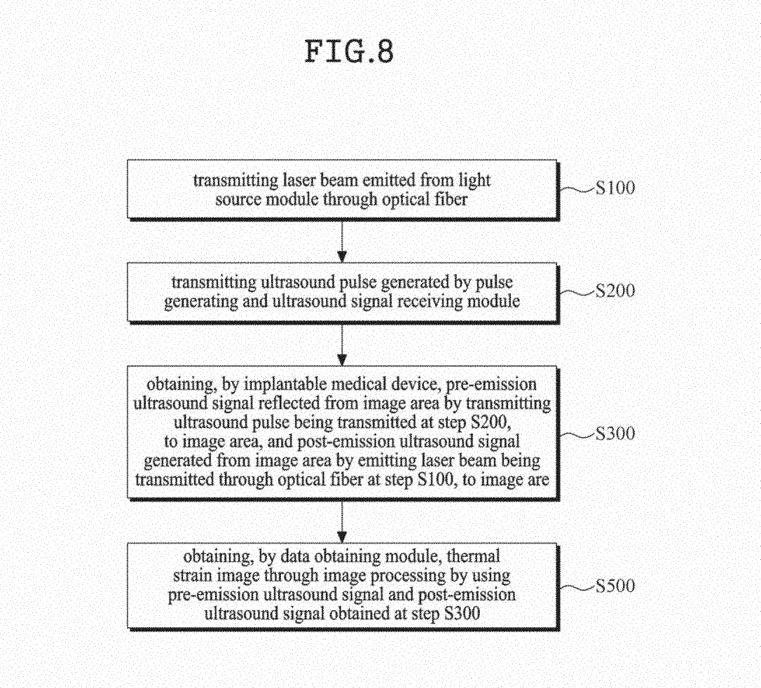

FIG. 8 is a flowchart illustrating a laser-induced thermal strain imaging method using an implantable medical device 330 according to an embodiment of the present invention. As shown in FIG. 8, the laser-induced thermal strain imaging method using the implantable medical device 330 according to the embodiment of the present invention may include: transmitting a laser beam emitted from the light source module 100 through the optical fiber 333 at step S100; transmitting an ultrasound pulse generated by the pulse generating and ultrasound signal receiving module 400 at step S200; obtaining, by the implantable medical device 330 at step S300, a pre-emission ultrasound signal reflected from an image area by transmitting the ultrasound pulse being transmitted at step S200, to the image area, and a post-emission ultrasound signal generated from the image area by emitting the laser beam being transmitted through the optical fiber at step S100, to the image area; and obtaining, by the data obtaining module 500 at step S500, an thermal strain image through image processing by using the pre-emission ultrasound signal and the post-emission ultrasound signal obtained at step S300.

Here, detailed configurations of the light source module 100, the pulse generating and ultrasound signal receiving module 400, the implantable medical device 330, and the data obtaining module 500 at steps S100, S200, S300, and S500 are the same as described above with reference to FIGS. 1 to 5, and thus description thereof will be omitted.

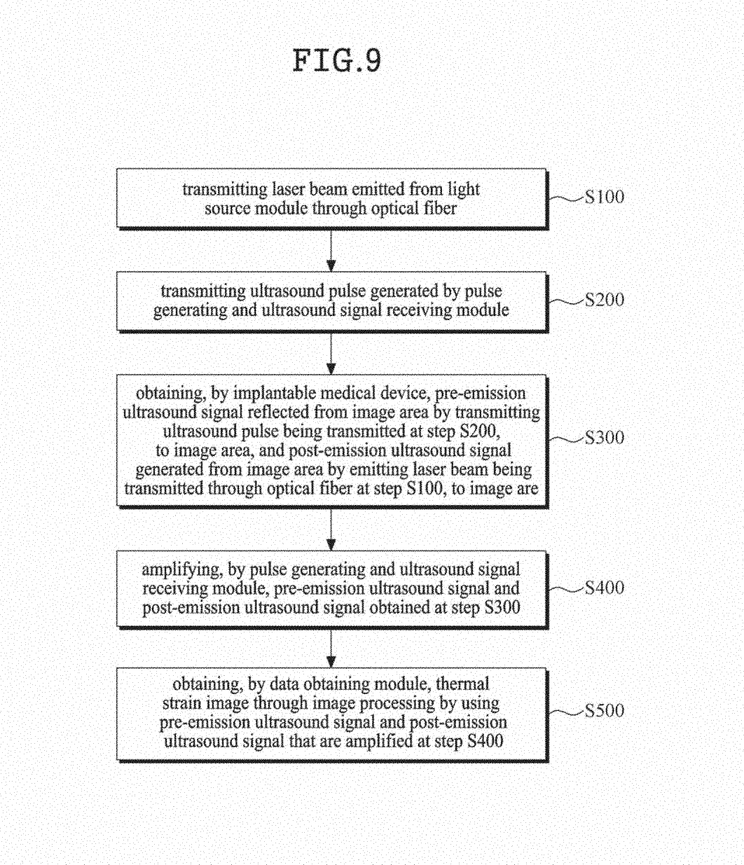

FIG. 9 is a flowchart illustrating a laser-induced thermal strain imaging method using an implantable medical device 330 according to another embodiment of the present invention. As shown in FIG. 9, according to another embodiment of the present invention, before step S500, the method may further include amplifying, by the pulse generating and ultrasound signal receiving module 400, the pre-emission ultrasound signal, and the post-emission ultrasound signal obtained at step S300.

Here, at step S500, the data obtaining module 500 may obtain the thermal strain image through image processing by using the pre-emission ultrasound signal and the post-emission ultrasound signal that are amplified at step S400.

In the meantime, detailed configurations of the pulse generating and ultrasound signal receiving module 400 and the data obtaining module 500 at steps S400 and S500 are the same as described above with reference to FIGS. 1 to 3D, and 5, and thus description thereof will be omitted.

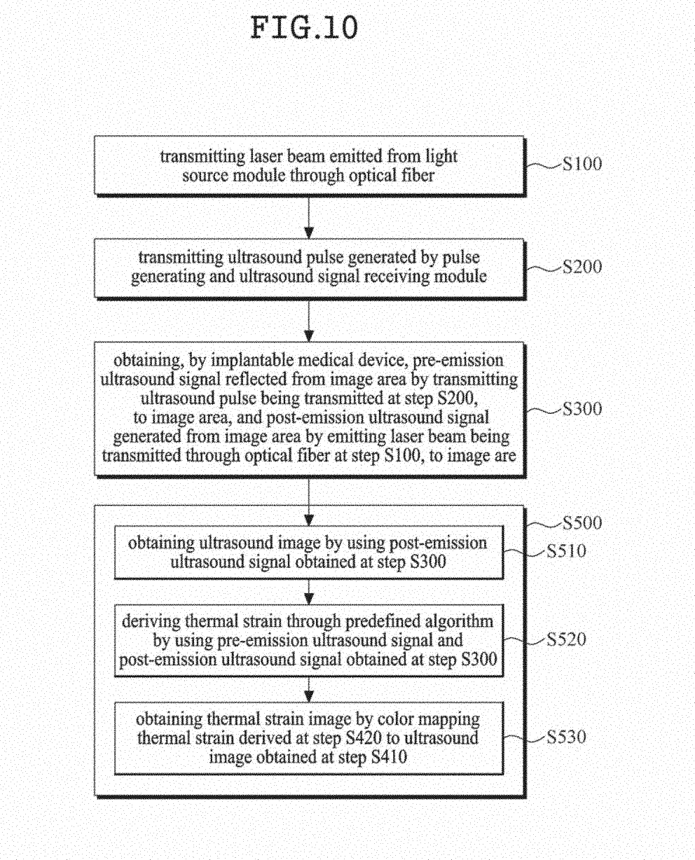

FIG. 10 is a flowchart illustrating a laser-induced thermal strain imaging method using an implantable medical device 330 according to still another embodiment of the present invention. As shown in FIG. 10, according to still another embodiment of the present invention, the step S500 may include: obtaining the ultrasound image at step S510 by using the post-emission ultrasound signal obtained at step S300; deriving thermal strain at step S520 through the predefined algorithm by using the pre-emission ultrasound signal and the post-emission ultrasound signal obtained at step S300; and obtaining the thermal strain image at step S530 by color mapping the thermal strain derived at step S420 to the ultrasound image obtained at step S410.

Here, detailed description of steps S510 to S530 are the same as described above with reference to FIGS. 3A to 3D, and 5, and thus description thereof will be omitted.

In the meantime, detailed configurations of the implantable medical device 330 for laser-induced thermal strain imaging according to an embodiment of the present invention are the same as described above with reference to FIGS. 3A to 3D.

Although the embodiments of the present invention have been disclosed for illustrative purposes, those skilled in the art will appreciate that various modifications and changes are possible, without departing from the scope and spirit of the invention as disclosed in the accompanying claims.

What is claimed is:

1. A laser-induced thermal strain imaging system using an implantable medical device, the system comprising:
a light source module emitting a laser beam;
a light concentrating module concentrating the laser beam emitted from the light source module through an objective lens so as to enable the laser beam to enter an optical fiber;
an ultrasound signal obtaining module provided with a scanning stage and the implantable medical device, the ultrasound signal obtaining module emitting the laser beam received through the optical fiber to an image area and obtaining an ultrasound signal generated from the image area;
a pulse generating and ultrasound signal receiving module generating an ultrasound pulse being transmitted to the ultrasound signal obtaining module, and receiving the ultrasound signal obtained by the ultrasound signal obtaining module and amplifying the ultrasound signal; and
a data obtaining module obtaining a thermal strain image through a predefined algorithm by using the ultrasound signal amplified by the pulse generating and ultrasound signal receiving module.

2. The system of claim 1, wherein the laser beam has a wavelength determined according to absorptance of the image area.

3. The system of claim 1, wherein the light concentrating module uses a collimator to transmit the laser beam emitted from the light source module to the objective lens without being dispersed.

4. The system of claim 1, wherein the scanning stage controls rotation and pull-back of the implantable medical device.

5. The system of claim 1, wherein the implantable medical device includes:
an ultrasound transducer obtaining the ultrasound signal generated from the image area;
the optical fiber having an end provided at a side of the ultrasound transducer, the optical fiber emitting the laser beam emitted from the light source module to the image area;
a pulse and ultrasound signal transmitting means coupled to the ultrasound transducer, the pulse and ultrasound signal transmitting means transmitting the ultrasound pulse generated by the pulse generating and ultrasound signal receiving module to the ultrasound transducer, and transmitting the ultrasound signal obtained by the ultrasound transducer to the pulse generating and ultrasound signal receiving module; and
a housing composed of a corrosion-resistant material made of metal to protect the ultrasound transducer, the optical fiber, and the pulse and ultrasound signal transmitting means from outside.

6. The system of claim 5, wherein the implantable medical device is covered with an ultra-thin chemical film to protect an inner structure thereof.

7. The system of claim 5, wherein the end of the optical fiber is provided with a prism attached thereto or is inclinedly cut so as to emit the laser beam to the image area.

8. The system of claim 5, wherein the ultrasound transducer obtains a pre-emission ultrasound signal and a post-emission ultrasound signal, the pre-emission ultrasound signal being reflected from the image area by transmitting the ultrasound pulse to the image area before the laser beam is emitted, and the post-emission ultrasound signal being emitted from the image area by absorbing the laser beam after the laser beam is emitted.

9. The system of claim 8, wherein the data obtaining module obtains the thermal strain image by color mapping thermal strain derived through the predefined algorithm by using the pre-emission ultrasound signal and the post-emission ultrasound signal, to an ultrasound image obtained by performing image processing on the post-emission ultrasound signal.

10. A laser-induced thermal strain imaging method using an implantable medical device, the method comprising:
(1) transmitting a laser beam emitted from a light source module through an optical fiber;
(2) transmitting a ultrasound pulse generated by a pulse generating and ultrasound signal receiving module;
(3) obtaining, by the implantable medical device, a pre-emission ultrasound signal reflected from an image area by transmitting the ultrasound pulse being transmitted at step (2), to the image area, and a post-emission ultrasound signal generated from the image area by emitting the laser beam being transmitted through the optical fiber at step (1), to the image area; and
(4) obtaining, by a data obtaining module, a thermal strain image through image processing by using the pre-emission ultrasound signal and the post-emission ultrasound signal obtained at step (3).

11. The method of claim 10, wherein the laser beam at step (1) has a wavelength determined according to absorptance of the image area.

12. The method of claim 10, wherein the implantable medical device includes:
an ultrasound transducer obtaining an ultrasound signal generated from the image area;
the optical fiber having an end provided at a side of the ultrasound transducer, the optical fiber emitting the laser beam emitted from the light source module to the image area;
a pulse and ultrasound signal transmitting means coupled to the ultrasound transducer, the pulse and ultrasound signal transmitting means transmitting the ultrasound pulse generated by the pulse generating and ultrasound signal receiving module to the ultrasound transducer, and transmitting the ultrasound signal obtained by the ultrasound transducer to the pulse generating and ultrasound signal receiving module; and
a housing composed of a corrosion-resistant material made of metal to protect the ultrasound transducer, the optical fiber, and the pulse and ultrasound signal transmitting means from outside.

13. The method of claim 12, wherein the implantable medical device is covered with an ultra-thin chemical film to protect an inner structure thereof.

14. The method of claim 12, wherein the end of the optical fiber is provided with a prism attached thereto or is inclinedly cut so as to emit the laser beam to the image area.

15. The method of claim 10, before step (4), further comprising:
amplifying, by the pulse generating and ultrasound signal receiving module, the pre-emission ultrasound signal and the post-emission ultrasound signal obtained at step (3).

16. The method of claim 10, wherein step (4) includes:
(4-1) obtaining an ultrasound image by using the post-emission ultrasound signal obtained at step (3);
(4-2) deriving thermal strain through a predefined algorithm by using the pre-emission ultrasound signal and the post-emission ultrasound signal obtained at step (3); and
(4-3) obtaining the thermal strain image by color mapping the thermal strain derived at step (4-2) to the ultrasound image obtained at step (4-1).

17. An implantable medical device for laser-induced thermal strain imaging, the device comprising:
an ultrasound transducer obtaining an ultrasound signal generated from an image area;
an optical fiber having an end provided at a side of the ultrasound transducer, the optical fiber emitting a laser beam emitted from an external light source to the image area;
a pulse and ultrasound signal transmitting means coupled to the ultrasound transducer, the pulse and ultrasound signal transmitting means transmitting an ultrasound pulse generated by an external pulser-receiver to the ultrasound transducer, and transmitting the ultrasound signal obtained by the ultrasound transducer to the external pulser-receiver; and
a housing composed of a corrosion-resistant material made of metal to protect the ultrasound transducer, the optical fiber, and the pulse and ultrasound signal transmitting means from outside.

18. The device of claim 17, wherein the implantable medical device is covered with an ultra-thin chemical film to protect an inner structure thereof.

19. The device of claim 17, wherein the end of the optical fiber is provided with a prism attached thereto or is inclinedly cut so as to emit the laser beam to the image area.

20. The device of claim 17, wherein the ultrasound transducer obtains a pre-emission ultrasound signal and a post-emission ultrasound signal, the pre-emission ultrasound signal being reflected from the image area by transmitting the ultrasound pulse to the image area before the laser beam is emitted, and the post-emission ultrasound signal being emitted from the image area by absorbing the laser beam after the laser beam is emitted.
